(19) United States
(12) Patent Application Publication
(10) Pub. No.: US 2021/0172839 A1
(43) Pub. Date: Jun. 10, 2021

(54) Devices And Methods For Cutting And Retrieving A Sample of Wood

(71) Applicants: Roger Thomas Young, Ebberston, Scarborough (GB); John Brian Lund, Kirby Misperton (GB); Julian Dyson, Pickering Yorkshire (GB)

(72) Inventors: Roger Thomas Young, Ebberston, Scarborough (GB); John Brian Lund, Kirby Misperton (GB); Julian Dyson, Pickering Yorkshire (GB)

(21) Appl. No.: 17/047,970

(22) PCT Filed: Apr. 15, 2019
(86) PCT No.: PCT/GB2019/051070

§ 371 Date: Oct. 15, 2020

(30) Foreign Application Priority Data
Apr. 15, 2018 (GB) 1806146.5

(51) Int. Cl. G01N 1/08; G01N 33/46; B23B 51/04

(52) U.S. Cl. CPC G01N 1/08; B23B 51/044; G01N 33/46

(57) ABSTRACT

A wood core sampling device includes a metal barrel having a cutting end and an opposite threaded end. The cutting end is shaped to have a smooth external protrusion and a smooth internal expansion cavity. The device can be hammered into a tree and removed with a slide hammer, the sample core breaking from the tree and being removed inside the barrel.

### CROSS-REFERENCE TO RELATED APPLICATIONS

This is the US national stage application of International Application No. PCT/GB2019/051070, filed Apr. 15, 2019, which claims the benefit of priority from GB Application No. 1806146.5, filed Apr. 15, 2018. These prior applications are incorporated by reference in their entirety.

### BACKGROUND

This invention relates to devices and methods for cutting and retrieving a sample of wood—for example, a solid cylinder in shape, from a live tree or from cut timber. Such samples are required for use in scientific research and in laboratory analysis. Conventionally, samples are taken from trees or timber by cutting cross sections from sawn trunks using a chainsaw, which is heavy and difficult to transport, or by drilling out solid cores by hand, which is slow and laborious.

W09711346 (Elliott et al.) discloses a wood coring drill bit intended to be driven by a rotary power drill. JP 2017226038 (Matsuo et al.) discloses a hole saw intended to be driven by a rotary power drill.

### BRIEF SUMMARY

Preferred embodiments of the present invention aim to provide generally improved devices and methods for cutting and retrieving a sample of wood.

According to one aspect of the present invention, there is provided a wood core sampling device comprising a metal tube having a circular cutting edge at one end, the wall thickness of the tube being arranged so that from the cutting edge the external diameter of the tube smoothly increases then decreases to form a protrusion and, at or adjacent the cutting edge, the internal diameter of the tube smoothly increases then decreases to form an expansion cavity.

Preferably, the end of the tube including said protrusion and the expansion cavity is carried on a removable threaded shank that in use co-operates with a thread on the metal tube.

Preferably, the end of the metal tube opposite to the cutting edge is threaded to cooperate in use with either a threaded plug or the threaded end of a slide hammer.

The threaded plug and/or slide hammer may be provided in combination with the wood core sampling device.

The said protrusion may be in the form of a collar, annulus or bulge.

In another aspect, the invention provides a method of cutting and retrieving a sample of wood by means of a wood core sampling device according to any of the preceding aspects of the invention, the method comprising the steps of driving the device into a body of wood with the cutting edge of the device leading, the device being driven by impact applied to the end of the metal tube opposite to the cutting edge; and subsequently retrieving the device from the body of wood, with the sample of wood contained within the device.

The step of retrieving the device from the body of wood is preferably effected by means of an extraction tool that engages the end of the metal tube opposite to the cutting-edge.

Preferably, said extraction tool comprises a slide hammer.

### BRIEF DESCRIPTION OF THE DRAWINGS

For a better understanding of the invention, and to show how embodiments of the same may be carried into effect, reference will now be made, by way of example, to the accompanying diagrammatic drawings, in which:

FIG. 1 is a perspective view of a wood core sampling device according to one embodiment of the present invention;

FIG. 2 illustrates use of the sampling device:

FIG. 3 is a sectional view of the sampling device;

FIG. 4 shows the device in partial cross-section in a tree, together with a slide-hammer used to extract the device from the tree:

FIG. 5 is a sectional view of the cutting end of the device: and

FIG. 6 is a part view of a variation.

### DETAILED DESCRIPTION

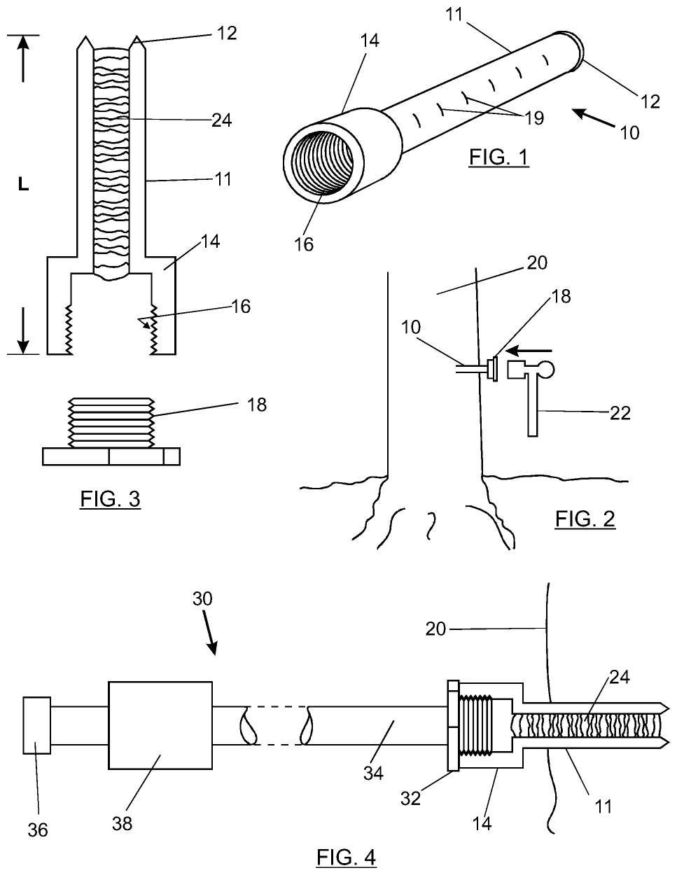

In FIG. 1, a wood core sampling device (or sampler) 10 comprises a cylindrical metal barrel 11 having a cutting end 12 and an opposite end 14 of increased diameter which has an internal thread 16. FIG. 3 shows a cut core of wood 24 within the barrel 11 of the sampler 10, and a threaded cap 18 which cooperates with the internal thread 16. In FIG. 2, the sampler 10 is driven into a tree 20 by blows from a hammer 22 on the threaded cap 18 when engaged with the internal thread 16.

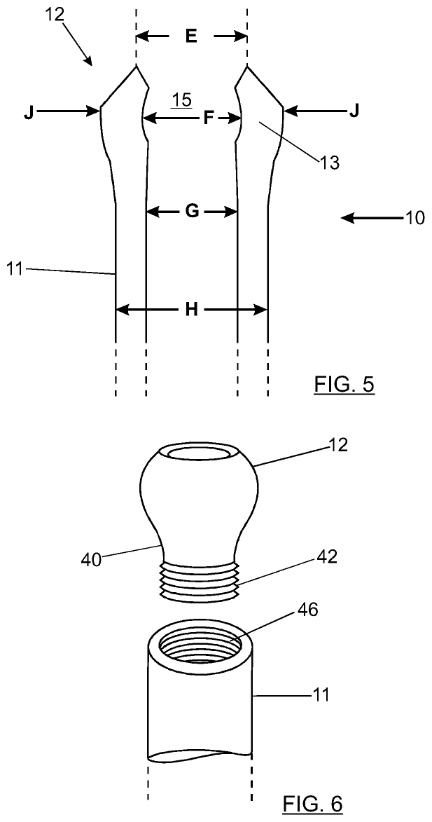

FIG. 5 shows the cutting end 12 of the sampler 10 in more detail. The cutting edge of the end 12 is circular and of diameter E. Adjacent to the cutting edge, the wall thickness of the barrel 11 increases. The external diameter of the barrel 11 increases smoothly to J, then decreases again smoothly to H for the remainder of the length of barrel 11. These diameter changes form a smooth circular protrusion 13, which may be in the form of a collar, annulus or bulge, and has the effect of reducing friction between the outside of the barrel 11 and the wood as the sampler 10 is hammered into the tree, in contrast to a tubular cutter of constant diameter. FIG. 5 also shows that as the wall thickness increases, the internal diameter of the barrel 11 close or adjacent to the cutting end 12 increases smoothly as shown at F, then decreases smoothly to G for the remainder of the length of the barrel 11. A circular expansion cavity 15 is thus formed. This cavity allows the cut plug of wood (24 in FIG. 3) to expand after it passes the cutting edge at end 12 and moves along the barrel 11. The cut plug 24 is equal in length to the length of barrel 11 hammered into the tree 20 and is initially still attached to the tree.

FIG. 4 illustrates the extraction of the sampler 10 from a tree 20 by use of a slide hammer 30 of conventional construction. When the sampler 10 has been hammered into the tree 20 and in so doing contains the required plug sample 24, the threaded plug 18 is unscrewed and replaced with the threaded end 32 of the slide hammer 30. The threaded end 32 is attached to a metal rod 34 which carries at the opposite end a stop 36. On the rod 34 is a slide bob 38. To remove the barrel 11 and plug 24, the slide bob 38 is manually advanced rapidly along the rod 34 to strike the stop 36, which impact results in a force in a direction which pulls the barrel 11 out of the tree 20. At the first impact (usually), the plug of wood 24 snaps off the tree 20, and subsequent impacts draw the barrel 11 out completely while retaining the plug 24. Referring now to FIG. 3, the sample 24 is removed by a suitable prod inserted into the cutting end 12, and retrieved at the thick end 14. The extracted core 24 is arranged to be of sufficient weight and length to satisfy analysis requirements, yet the tree has not been harmed or felled.

It has been found that a sample 24 can be taken in a few minutes. The provision of the protrusion 13 also reduces friction while the slide hammer is in use, so that the weight and length of the slide hammer parts can be of practical dimensions.

In contrast to prior known devices, it is not necessary to rotate the sampler 10 in use (although this can be done if desired). A regular hammer 22 can be used to drive the sampler 10 into the tree 20 (or other timber). A slide hammer 30 can be used to remove the sampler 10 with core 24.

It will be understood that the edge of the cutting end 12 will become blunt with repeated use. FIG. 6 shows a variation in which the cutting end 12 is formed on a short shank 40 that is threaded at 42. The end of barrel 11 carries a cooperating thread 46 so that the cutting end can be replaced. This also allows barrels 11 of different lengths to be used.

Different lengths of sample plug will be required in different circumstances, so the barrel 11 is marked with measuring lines 19 (FIG. 1).

In this specification, the verb “comprise” has its normal dictionary meaning, to denote non-exclusive inclusion. That is, use of the word “comprise” (or any of its derivatives) to include one feature or more, does not exclude the possibility of also including further features. The word “preferable” (or any of its derivatives) indicates one feature or more that is preferred but not essential.

All or any of the features disclosed in this specification (including any accompanying claims, abstract and drawings), and/or all or any of the steps of any method or process so disclosed, may be combined in any combination, except combinations where at least some of such features and/or steps are mutually exclusive.

Each feature disclosed in this specification (including any accompanying claims, abstract and drawings), may be replaced by alternative features serving the same, equivalent or similar purpose, unless expressly stated otherwise. Thus, unless expressly stated otherwise, each feature disclosed is one example only of a generic series of equivalent or similar features.

1. A wood core sampling device comprising a metal tube having a circular cutting edge at one end, a wall thickness of the tube being arranged so that from the cutting edge an external diameter of the tube smoothly increases then decreases to form an annular protrusion and, at or adjacent the cutting edge, an internal diameter of the tube smoothly increases then decreases to form an expansion cavity adjacent the cutting edge, the sampling device being arranged to be driven by impact into a body of wood with the cutting edge leading.

2. A wood core sampling device according to claim 1 in which an end of the tube including said protrusion and the expansion cavity is carried on a removable threaded shank that in use cooperates with a thread on the metal tube.

3. A wood core sampling device according to claim 1 in which an end of the metal tube opposite to the cutting edge is threaded to cooperate in use with either a threaded plug or a threaded end of a slide hammer.

4. A wood core sampling device according to claim 3, in combination with said threaded plug and slide hammer.

5. (canceled)

6. A method of cutting and retrieving a sample of wood using a wood core sampling device according to claim 1, the method comprising the steps of driving the device into a body of wood with the cutting edge of the device leading, the device being driven by impact applied to an end of the metal tube opposite to the cutting edge; and subsequently retrieving the device from the body of wood, with the sample of wood contained within the device.

7. A method according to claim 6, wherein the step of retrieving the device from the body of wood is effected using an extraction tool that engages the end of the metal tube opposite to the cutting-edge.

8. A method according to claim 7, wherein said extraction tool comprises a slide hammer.
